(19) United States
(12) Patent Application Publication
(10) Pub. No.: US 2009/0170199 A1
(43) Pub. Date: Jul. 2, 2009

(54) SEPARATION AND SORTING OF DIFFERENT BIOLOGICAL OBJECTS

(75) Inventors: Ryan Stephen RAZ, Toronto (CA); Anthony PRESTA, Hamilton (CA); Laurent SAFAR, Paris (FR); Frederic REVAH, Paris (FR)

Correspondence Address: FOLEY AND LARDNER LLP;SUITE 500, 3000 K STREET NW, WASHINGTON, DC, 20007, US

(73) Assignees: CANOPUS BIOSCIENCE LIMITED; HERA DIAGNOSTICS

(21) Appl. No.: 12/142,696

(22) Filed: Jun. 19, 2008

Related U.S. Application Data
(63) Continuation-in-part of application No. 11/334,303, filed on Jan. 18, 2006.
(60) Provisional application No. 60/643,972, filed on Jan. 18, 2005.

(51) Int. Cl. C12N 5/00

(52) U.S. Cl. 435/378

(57) ABSTRACT

The present invention relates to a method for the separation of biological objects in a solution which have different viscoelastic properties, wherein said method comprises a filtration step allowing the higher viscoelastic biological objects to pass through the membrane while retaining the lower viscoelastic biological objects above the membrane, and a recovery step wherein the separated lower viscoelastic biological objects are recovered above or onto the membrane and/or the separated higher viscoelastic biological objects are recovered in the filtrate. Advantageously, the biological objects are cells. More advantageously, the recovered cells are viable cells. In one preferred embodiment, the cells are tumor cells. In another preferred embodiment, the cells are fetal cells and the method finds an application in prenatal diagnosis.

The present invention relates to a method for the separation of biological objects in a solution which have different viscoelastic properties, wherein said method comprises a filtration step allowing the higher viscoelastic biological objects to pass through the membrane while retaining the lower viscoelastic biological objects above the membrane, and a recovery step wherein the separated lower viscoelastic biological objects are recovered above or onto the membrane and/or the separated higher viscoelastic biological objects are recovered in the filtrate. Advantageously, the biological objects are cells. More advantageously, the recovered cells are viable cells. In one preferred embodiment, the cells are tumor cells. In another preferred embodiment, the cells are fetal cells and the method finds an application in prenatal diagnosis.

It is often desirable to examine biological samples, and specimens for signs of abnormality and disease.

As an example, the cells in a sample of blood or spinal fluid might need to be examined for indications of cancer. Because these types of samples might well contain millions of cells, it is very advantageous to separate the majority cells and fluids that are not of interest, thus concentrating the cells of interest.

In blood and spinal fluids it is desirable to remove plasma, erythrocytes red blood cells, and leukocytes (white blood cells), thus concentrating the small number of cells that are not normally present and that might exhibit signs of abnormality such as cancer. As leukocytes are often very similar to the cells of interest it is difficult to remove these cells without losses. The resulting concentrated cells of interest are then used for further analysis.

The methods currently available for separating cell types comprise separation by size, separation by centrifugation (density/specific gravity), and separation relative to the chemical or biochemical properties.

Separation by centrifugation works well when the two types of cells are very different as in the example of the separation of white and red blood cells. But centrifugation fails when the two types of cells have similar density and size, such as white blood cells and cancer cells. A further limitation of centrifugation-based cell separation is that the density of the cells are not constant, as even dead cells react to the conditions of their surrounding and environment.

Separation by (bio)chemical properties utilizing immuno-based chemistry by antibody binding of the cell to a surface antigen (which can possibly be attached to magnetic beads) is expensive, labor-intensive, and time-consuming. Many of the steps can have cell losses thus reducing the separation efficiency of this type of method. Also, cells will be lost if they don't have the matching antigen, and/or if the antigen is obscured by other blood components. Blood plasma proteins may coat the cells in circulation (a possible method of cancer cells evading the immune system) thus preventing their recognition by the antibody. The cells separated by this method are often in a form that is difficult for a visual examination of the results.

Separation by size is usually done by filtering through a filter, or an array of one or more hollow tubes with a specific hole size. Cells that are larger than the hole stay on one side of the filter while smaller cells go through the filter and are collected on the other side of the filter. In this separation method, a fixative agent is used for stabilizing the membrane of the cells, such as formaldehyde. However, cells are no more viable after the action of fixative agents, and cannot be cultured. Moreover, if the two types of cells have an overlapping size distribution (a certain portion of the cells of one type are larger while another portion are smaller than the other type of cell), then the filter does not separate the two types effectively, resulting in a loss of some of the cells of interest thus reducing separation efficiency.

Consequently, separation by the above methods can damage the cells both bio-chemically, and mechanically, thus changing the cell morphology, and inhibiting subsequent processing and analysis.

There is thus a need for a method which allows the separation of different biological objects which may have the same size or an overlapping size distribution and which, advantageously, does not denature said biological objects such that, in the case of cells, the separated cells are viable and can be further cultured.

The Inventors have elaborated a new method of separation which meets the need in the art. According to this method, the biological objects are separated relative to their different biological properties, even if their size is the same or overlaps. Moreover, this method allows advantageously the recovered biological objects to be further used for culture applications.

Accordingly the present invention relates to a method to separate the objects by object type where the different object types can not be fully differentiated by size, shape, and density (leukocytes and certain cancer cells are two important examples). This method also provides a high separation efficiency, which allows the use of smaller sample sizes with less risk of missing objects of interest. Moreover, without any damage to the objects of interest, both biochemically, and morphologically, this method does not interfere with subsequent processing and analysis, and the correct morphology of the resulting objects is maintained. No chemical/biochemical preparation of the objects of interest is used (all such known per se preparations modifying biochemical, and/or biophysical and/or morphological properties of such objects). The separated objects can then be easily presented on a slide in a way that is preferred by a pathologist, or be read by automated vision system, or remain in a liquid solution for subsequent processing.

Thus, the subject-matter of the present invention is a method of separating multiple natural biological objects in a solution, wherein the biological objects are composed of at least a natural lower viscoelastic biological object type and a natural higher viscoelastic biological object type, and the natural lower viscoelastic biological objects have lower viscoelastic properties than the natural higher viscoelastic biological objects, wherein the natural lower viscoelastic biological objects are at least one of the group consisting of circulating fetal cells and tumoral or cancer cells, the method comprising:
a filtration step of the solution wherein:
   the membrane is porous and the diameter of the pores is less than the diameter of the natural lower viscoelastic biological objects and also less than the diameter of a portion of the natural higher viscoelastic biological objects, and allowing the natural higher viscoelastic biological objects to pass through the membrane while retaining the natural lower viscoelastic biological objects above the membrane, and
   a controlled force is applied, which is kept lower than the predetermined force needed to force the natural lower viscoelastic biological objects to pass through the membrane, and which is higher than or equal to the predetermined force needed to force the natural higher viscoelastic biological objects to pass through the holes, and
a recovery step wherein the separated natural lower viscoelastic biological objects are recovered above or onto the membrane and/or the separated natural higher viscoelastic biological objects are recovered in the filtrate.

It must be understand throughout the whole application that the term “natural” is used to qualify an object or a property that is not chemically/biochemically modified between the sampling and the recovery step of the method according to the invention.

The biological objects may be of any type, such as cells, bacteria, viruses, and yeasts, such list being not limiting. The biological objects in solution may be obtained from any biological sample. The biological sample may be bodily fluids, such as blood, spinal fluids, urine, any tissue and tumor biopsies. The method is not limited to a liquid biological sample. As an example a solid tissue biopsy can be preprocessed to break the tissue down into individual cells. The cells can then be suspended in a preservative fluid. It may also be water and soil samples, plant tissues and fluids, etc. . . .

Advantageously, the multiple biological objects are at least two cell types.

The cells to be separated may be of any type. These can be cells naturally present in the blood such as megakaryocytes, monocytes, macrophages, dendritic cells, neutrophil granulocytes, eosinophil granulocytes, basophil granulocytes, mast cells, helper T cell, suppressor T cell, cytotoxic T cells, B cells, natural killer cells, reticulocytes, stem cells and committed progenitors for the blood and immune system. Cells to be separated can also belong to other origins. They can be epithelial cells (keratinizing epithelial cells, wet stratified barrier epithelial cells, exocrine secretory epithelial cells), cells from the gut, exocrine glands and urogenital tract, endothelial cells, metabolism and storage cells (hepatocyte, white fat cell, brown fat cell, liver lipocyte), barrier function cells (lung, gut, exocrine glands and urogenital tract), epithelial cells lining closed internal body cavities, extracellular matrix secretion cells, contractile cells, sensory transducer cells, autonomic neuron cells, sense organ and peripheral neuron supporting cells, central nervous system neurons and glial cells, lens cells, pigment cells, germ cells, nurse cells.

The cells to be separated could also be diseased cells such as mutant, virally infected cells or tumor cells and belong to any of the above cell types.

In a preferred embodiment, the cell types which are recovered are destined to be analyzed by biological, genetic, immunohistochemical and biochemical methods after further division and expansion in culture. Accordingly, the cells recovered are advantageously viable cells and the solutions used for the filtration step do not contain any reagent that kills the isolated cells.

In another embodiment, the cell types which are recovered are processed for further analysis by biological, genetic and biochemical, or immunohistochemical, methods without further expansion in culture.

In another embodiment, after recovery of the target cells onto the membrane, their nucleic acid material is extracted for further analysis. The acid nucleic (DNA, RNA) may allow the identification of genetic defects or genes specifically expressed in the target cells.

The cytoskeleton is a three-dimensional polymer scaffold which spans the cytoplasm of eukaryotic cells. This network is mainly composed of actin filaments, microtubules, intermediate filaments, and accessory proteins. It provides the cell structure and affects cell motility as well as viscoelastic properties. The viscoelastic properties of cells determine the degree of cell deformation as a result of mechanical forces and, consequently, affect cellular structure and function. The determination of the viscoelastic properties of living cells requires the quantification of force versus strain relationship of cells under physiological conditions. Several papers describe the techniques which can be used to determine the viscoelastic properties, and are well known by the man skilled in the art.

Numerous systems have been described in order to measure the rheological/viscoelastic properties of cells, including micropipette aspiration (Evans E. and Yeung A., Biophys. J. (1989), 56, 151-160.), passage through the micro holes of a membrane (Frank R. S., Tsai M. A. J Biomech Eng. (1990); 112, 277-82), optical tweezers (Ashkin A. and Dziedzic J. M., Proc. Natl. Acad. Sci. USA (1989), 86, 7914-7918), Atomic Force Microscopy [Benoit M. et al., Nature Cell Biol. (2000), 2, 313-317). These techniques can be coupled to an adequate Theological model.

Micropipette aspiration technique is a frequently used method to measure viscoelastic properties of cells. This technique has the advantage to measure viscoelastic properties in solution and in the physiological environment of the cells. As an example, viscoelastic of both hepatocytes and hepatocellular carcinoma (HCC) cells were measured by means of a micropipette aspiration technique (Wu ZZ and al., Biorheology, 2000, 37, 279-290).

According to the present invention, during the first step of the process, cells are sorted according to their natural viscoelastic properties by a filtering step across a membrane containing holes with an appropriate size. Preferably, a controlled force is applied during the filtration step, which is kept lower than the predetermined force needed to force the natural lower viscoelastic biological object to pass through the membrane, and which is higher than or equal to the predetermined force needed to force the natural higher viscoelastic biological object to pass through the holes.

The expression “controlled force”, according to the invention, is used to designate the force applied during the filtration step to force the natural higher viscoelastic biological objects to pass through the membrane, containing holes with an appropriate size, while retaining the natural lower viscoelastic biological objects, and preserving the integrity of the cells to be isolated.

This applied force results from a differential pressure created between the both side of the filtrating membrane, with force=(differential pressure)×(biological object surface). On another embodiment according to the invention, this applied force results from the acceleration applied by centrifugation, for example, on the biological objects, with force=acceleration×(biological object mass)

In the present invention, isolation of circulating cells of interest, starting from a peripheral blood sample, is based on the difference in viscoelastic properties between leukocytes cells and the circulating cells of interest. In a particular embodiment, the natural lower viscoelastic cells are tumor or cancer cells or fetal cells.

The membranes used in the present invention display hydrophobic properties and are mostly inert and strong, resulting in a constant pore size even when under pressure. The membranes used in the present invention are, for example, polycarbonate membranes. Polycarbonate membranes have the properties described above and a highly efficient cell transfer rate of isolated cells from the membrane to the glass slide used for its biological characterization.

In a particular embodiment, the natural lower viscoelastic cells are tumor or cancer cells. In a second particular embodiment, the natural lower viscoelastic cells are fetal cells. Preferably, the fetal cells are fetal cells circulating in maternal blood. Preferably, the controlled force which is applied during the filtration step corresponds to a force resulting from a differential pressure between 20 kPascals and 190 kPascals, advantageously between 40 kPascals and 60 kPascals, and more advantageously between 45 kpascals and 55 kPascals, and the average diameter of the pores is comprised between 3 μm and 15 μm, advantageously between 6 and 10 μm, and more advantageously between 8 and 10 μm, thus allowing to recover the tumor cells or the fetal cells on or above the membrane. A good adequacy between the pore size and the controlled force applied is desirable. In alternative, the filtration step is realized under a temperature comprised between 20° C. and 40° C.

Indeed, normal, fetal and cancerous circulating cells display different viscoelastic properties. Scientific literatures indicate that the most of leukocyte cell types have folded membranes. The unfolding of the membrane gives leukocytes viscoelastic properties that allow the cell when under pressure to elongate and to pass through a micropipet tip without damage even when the tip is less than ¼ the diameter of the leukocyte (E Evans and A Yeung, (1989). Biophysical Journal 56: 151-160). Neutrophils, whose diameter size is comprised between 10 and 12 μm, can be made to pass through 3 μm holes. In details, the different types of leukocytes are::
Small Lymphocytes:
   Represent 20-25% of the leukocytes, and have a diameter of 6-8 μm, a nucleus spheroid or ovoid, chromatin in dense lumps, cytoplasm scarce and stained pale blue,
Medium Lymphocytes:
   Represent 1.5-2.0% of the leukocytes and have a diameter of 8-12 μm, chromatin less dense, more cytoplasm and tend to surround more of nucleus
Neutrophils:
   Represent 60-70% of the leukocyte and have a diameter of 10-12 μm, a nucleus with 2-8 lobes, chromatin in dense coarse lumps, cytoplasm is acidophilic with neutrophilic granules and ‘drumstick’ on lobe in 3% of neutrophils in females
   1-2% of neutrophils are horse-shoe shaped nucleus and cytoplasm has granules.
Monocytes:
   Represent 3-8% of the leukocytes are largest leukocyte and have a diameter of 20 μm and a nucleus indented and pale cytoplasm abundant and basophilic, a non-uniform (foamy) appearance cytoplasm that may contain a few fine azurophilic granules.
Eosinophils:
   Represent up to 5% of the leukocytes and have a diameter of 12-15 μm, a nucleus less lobed, usually only bilobed, chromatin clumped but not as dense as in neutrophil, and a cytoplasm filled with numerous large eosinophilic (acidophilic) granules which stain pale-pink.
Basophils:
   Represent less than 1% of the leukocytes and have a diameter of 14 μm, a nucleus large and bilobed, chromatin that is more finely textured so nucleus is more pale stainingand a cytoplasm filled with large dark-blue staining granules (basophilic) which may obscure nucleus (Blackberry appearance).

Other types of cells lack this folded membrane and therefore have difficulty passing through a hole of less than the diameter of the cell. The smaller the membrane pore size in relation to the leukocyte size the greater the differential pressure is needed to force the leukocyte through the hole. For example, as the morphology of cell progresses from normal to cancer cell, the membrane changes in some cases getting thicker and in other cases getting thinner (Gang Zhang et al., 2002, World J Gastroenterol; 8(2), 243-246). When the cell membrane gets thinner it is more susceptible to damage and lyses. The damage threshold for the cells of interest puts an upper limit on the pressure differential which can be applied across the membrane without damaging the cells of interest. It thus seems that malignant transformation induces a decrease in viscoelastic properties.

The method of this patent is an effective method to separate cell types and relies on the difference in viscoelastic properties between different cells.

As an example, in the case of separation of circulating cells according to their viscoelastic properties within a blood sample, a polycarbonate membrane with conservative 8 μm pore size applying a differential pressure of 40 to 60 kPascals (In Custom cut from sheets 8 μm Whatman polycarbonate Nuclopore membranes) can be used under a temperature between 20° C. and 40° C. Because of their viscoelasticity, this will suffice to pass the majority of leukocytes although the size of 75% of those cells is larger than 8 μm. On the other hand other cells, i.e. not leukocytes, larger than 8 μm in diameter will be blocked.

For separating leukocytes from other types of cells that are smaller than 8 μm in diameter, a membrane as small as 4 μm can be used with the corresponding need for higher pressures only being limited by the damage threshold for the cell or by forcing the cell types of interest through the membrane. Polycarbonate membranes are used because they are hydrophilic, mostly inert, and strong with low elasticity resulting in the pore size remaining constant even when under pressure. Also polycarbonate membranes have a highly efficient cell transfer rate from the membrane to a glass slide.

Preferably, the solution containing the cell types is a mononuclear cell fraction which results from a centrifugation step of a blood sample. In a particular embodiment, the lower viscoelastic cells are circulating tumor cells.

In another embodiment, the at least one of the cell types is a fetal cell type. Preferably, the fetal cells are fetal cells circulating in maternal blood.

Fetal cells are present in the maternal circulation. Successful isolation of fetal cells from maternal blood will open new routes to replace invasive prenatal diagnosis methods (chorionic villus sampling or amniocentesis) with their inherent risks to the mother and fetus by non-invasive methods followed by genetic analysis on fetal cells (FISH, PCR, sequencing). Three different fetal cells are known to circulate in maternal blood: trophoblasts, fetal leukocytes and fetal erythrocytes (for review see Bianchi, British Journal of Haematology, 1999).

Fetal trophoblast cells, located outside the villus (extravillous) migrate during the first trimester into the maternal tissue of the placental bed. This process of invasion is unique to trophoblast cells and induces vascular adaptation of the maternal spinal arteries. As a consequence, a specific subset of trophoblast cells appears in the maternal blood as a normal feature (for review see Oudejans et al. 2003, Prenatal Diagnosis). The first wave peaks around the middle of the first trimester, the second wave peaks at the end of the first trimester.

A second aspect of the invention is an in vitro prenatal diagnosis comprising the method according to present invention wherein the at least one of the two cell types is a fetal cell type, as described above.

If we considered that in one milliliter of blood, there are 7 millions leukocytes, the number and size of the different type of leukocytes is described as follow:
Small lymphocytes: 6 to 8 μm in size, 1 575 000/ml, represent 22.5% of the leukocytes
Medium lymphocytes: 8 to 12 μm in size, 1 225/ml, represent 0.02% of the leukocytes
Neutrophil: 10 to 12 μm in size, 4 620 000/ml, represent 66% of the leukocytes
Monocyte: 20 μm in size, 385 000/ml, represent 5.5% of the leukocytes
Eosinophil 12 to 15 μm in size, 350 000/ml, represent 5% of the leukocyte
Basophil: 14 μm in size, 70 000/ml, represent 1% of the leukocytes
Fetal cells: approximatively 12 μm in size, 5/ml, represent 0.00007% of the total number of leukocytes.

We will see that the method according to the invention is very adapted for separation of the fetal cells circulating in the maternal blood through annexed examples.

While the above description contains many specificities, these should not be construed as limitations on the scope of the invention, but as exemplifications of the presently preferred embodiments thereof. Many other ramifications and variations are possible within the teachings of the invention.

### LEGEND OF THE FIGURES

FIGS. 1a, 1b, 1c, 1d: Closely related conceptual drawings of a cell being forced through a smaller hole by a pressure differential or a centrifugation force.

FIG. 2a is a schematic representation of two cell populations with overlapping size distribution. Cell population A has a larger mean size than cell population B. On the other hand, cell population A has higher viscoelastic properties than cell population B.

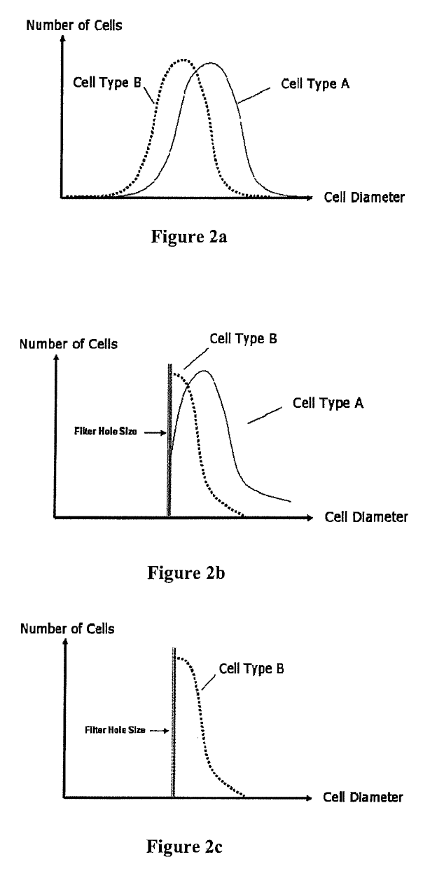

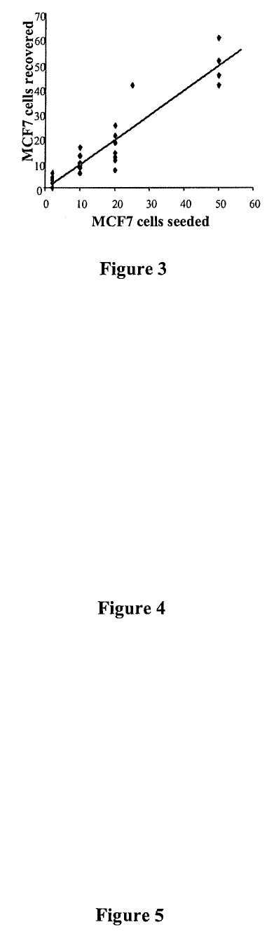

In the case of FIG. 2b the mixture of cell population A+ cell population B has been processed by classical separation by conventional size filtration onto a membrane. The scheme indicates the distribution of cells remaining above or onto the membrane. Cells with sizes smaller than the filter pore hole size will remain above or onto the membrane. Note the large amount of overlap between the two remaining populations after filtration. With a smaller hole size, a larger amount of type A cells remain with the type B cells, thus reducing the concentration of the cells of interest (cell type B). Conversely with a larger hole size, more type B cells (the cells of interest) are lost, reducing overall sensitivity to type B cells. Also, the position and shape of the distribution curves will vary from patient to patient. It is because of this overlap and variation in distribution that conventional filtering by size does poorly on the separation of the two cell types.

In the case of FIG. 2c the mixture of cell population A+ cell population B has been processed using the principle of the present invention using a controlled pressure differential to improve recovery and enrichment.

Cells of type A even when they are larger then the hole (pore) size of the membrane will pass through because of their higher viscoelastic properties as compared to type B cells. Type B cells will not get through the holes membrane unless the cell size is less than or close to the pore size of the membrane.

FIG. 2c represents the distribution of cells remaining above or onto the membrane using the principle of the present invention.

Note the high efficiency of separation in FIG. 2c as compared to FIG. 2b.

FIG. 3: MCF7 cells recovered using the present method from blinded samples seeded with MCF7 cells.

FIG. 4: Filtration device (see Examples—step 6.e) of Appendix A)

FIG. 5: Recovery of the cells from the membrane (see Examples—step 10 of Appendix A)

### DETAILED DESCRIPTION OF THE FIGURES

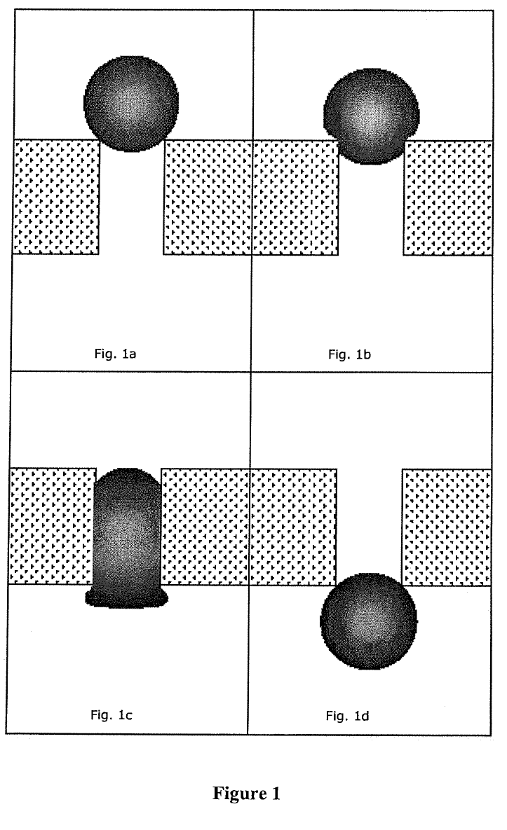

FIGS. 1a, 1b, 1c, 1d are conceptual drawings of a cell being forced through a smaller hole by the pressure differential of the centrifugation force.

a) Cell is attracted to empty hole by fluid flow through the hole. (higher pressure on the top or centrifugation force) FIG. 1a
b) Pressure differential or centrifugation force starts to deform and fold cell pushing it into the hole. FIG. 1b
c) Cell is pushed through the hole by pressure differential or centrifugation force FIG. 1c
d) Cell is expelled away from the hole by fluid flow through the hole FIG. 1d

The force (pressure differential or centrifugation) needed to push the cell through the smaller hole is dependent on size and the viscoelastic properties of the cell. Viscoelastic properties of an object are the properties that allow the object to elastically fold, and to bend, and to distort their shape, and to flow through holes and passageways that are smaller than the object. Literature indicates that the white blood cells (leukocytes) have relatively high viscoelastic properties; this allows them to flow through small diameter passageways and reach tissues via the body's microscopic blood vessels.

Tumor or cancer cells and fetal cells from the maternal blood can be of a similar size to that of white blood cells. But tumor or cancer cells and fetal cells are found to have considerably natural lower viscoelastic properties. Hence a tumor or cancer cell or a fetal cell needs considerably more force to push it through a small diameter hole as compared to a white blood cell of a similar size. The tumor or fetal cells will be stopped by the small hole size and will not go past the point in FIG. 1a. Exploiting this difference in the viscoelastic properties of the two cell types enables the cells to be separated by type. Sorting cells by utilizing this property is a unique method and the basis of this invention.

### EXAMPLES

### A—Example #1

### Tumoral or Cancer Cells

The protocols outlined below describe the method to isolate cancer cells from human blood samples. The samples are either taken from cancer patients, with the objective of isolating endogenous patient circulating tumor cells. Alternatively, as an experimental model for the validation of the present invention, cultured tumor cells are seeded into blood samples from healthy volunteers. In this latter setting the objective is to assess the yield and sensitivity of the isolation procedure.

A similar protocol can be used for the purification of fetal cells from maternal blood.

1. Equipment & Reagents
Cultured carcinoma cells
Becton-Dickinson Vacutainer tube (4 mL) with purple top (EDTA anti-coagulant)
Phlebotomy personnel for the safe collection of blood from human subjects
Ethanol (40% and 60%) in wash bottles
Clean Glass microscope slides non-coated or coated (recommended fresh Erie Scientific Superfrost Plus slides, follow manufacture's guidelines for storage of open boxes of slides)
Ficoll-Paque differential centrifugation medium (Amersham Bioscience #17-1440-02) brought to room temperature.
Centrifuge tubes capable of holding >12 mL (recommend the 15-mL Falcon conical bottom tubes), and
Swinging-bucket centrifuge capable of reaching speed specified for use of Ficoll-Paque product used for separation of mononuclear cell fraction from peripheral blood.
Fine curved non-serrated tip tweezers for handling membranes.
5 mL syringes×2 slip tip without tips (BD—ref 301603)

The following items need frequent washing (for large processing runs it is recommended that more are purchased). Includes 2 each of:

Manufacturer Kimble—Kontes

953701-0000 Glass Funnel top, 25 mm, 15 mL
953702-0001 Fritted Glass Support Base (it is anticipated that in future versions of device the glassware will be replaced by disposable)
consumable kit #1 containing:
   Membrane (Custom cut from sheets 8 μm Whatman polycarbonate Nuclopore filters); Sponge (Custom cut from sheets of hydrophilic polyurethane foam rubber produced by Lendell manufacturing); 10 cm silicon-tubing syringe tip; 12 cm silicon-tubing syringe tip (The length of the tip depends on the shape and length of the centrifuge tubes being used. Other materials such as hard plastics and stainless steel could also be used for the tip).
Sample fixative (>95% ethanol recommended)
Immunostaining reagents and equipment
In another version of the invention, the glassware can be replaced by disposable single-use plastic ware. The washing steps are then avoided.

2. Seeding Method

Cultured cancer cells (preferably a cell line that is not overly prone to clumping) are harvested according to usual cell biology procedures, e.g. cell containers are washed, detached by trypsinization for a suitable length of time, and then collected by centrifugation.

The collected cells are resuspended and washed in a 90% culture medium/10% serum solution (solution A), then centrifuged again for collection.

The cell density (cells/volume) is determined for the stock using a hemocytometer, taking a known volume from the well-dispersed stock.

A 4-mL whole blood sample is collected from the peripheral circulation of a healthy volunteer. The blood is collected in a commercial Vacutainer® with EDTA as the anticoagulant.

Cultured cancer cells are then added to the blood sample at a known nominal value by serial dilution of the dispersed stock. Solution A is used as the diluant throughout the series.

At every step in the series, and in the final seeded blood sample, the tube is gently mixed for cell dispersal.

The nominal value represents the approximate number of cells seeded into the sample; the exact desired number of cells cannot be achieved using the serial dilution method because of heterogeneity of the cell mixture. For exact seeding values (especially at low cell numbers), methods such as micromanipulation or flow cytometry are recommended.

3. Enrichment Method
The blood is transferred from the collection tube to a suitable centrifuge tube.
Slowly inject into the bottom the centrifugation tube 3.0 mL of Ficoll-Paque gradient centrifugation media at room temperature using the 5 mL syringe with the 12 cm tip.
The blood samples are centrifuged at a speed of 400 g for 30 minutes, using a partial or no brake at the end of the run. Batch size should be determined by the total batch processing time of 30 minutes excluding centrifugation time. It is estimated the maximum size of a batch should be from 4 to 6 samples.
The mononuclear cell fraction (or buffy coat) is aspirated from the centrifuge tube by immersing the tip of a 5-mL syringe fit with the 10 cm silicon-tubing tip attachment below the level of the buffy coat. Aspirate in a steady manner until a small amount of serum is aspirated. The tip should be lowered slightly and aspiration should continue until once again a small amount of serum is aspirated.
The aspirated buffy coat is added directly onto the membrane, pre-primed with 40% ethanol, and with about 10 mL of 40% ethanol remaining in the top chamber of the apparatus. Flush the syringe out by aspirating some of the fluid back into the syringe and back out again.
Filter the contents down to approximately 3 mL remaining in the top chamber. Wash the sample by addition of 10 mL of 40% ethanol and back flushing the membrane. Repeat as necessary until filtration is complete (the filtrate is clear and the flow rate is constant). With some samples the flow rate through the membrane may become very slow necessitating a back flush before the contents have reached the 3 mL mark.
Filter down to about the 1 mL mark and then slowly filter the contents until the liquid is just removed from above the membrane; do not allow the membrane to dry out.
After enrichment of the disseminated cancer cells, the cells are deposited on a slide by removing the membrane from the apparatus, placing the filter cell side up on a sponge minimally saturated with 60% ethanol. A microscope slide is pressed on the sponge such that the membrane is ‘sandwiched’ between glass microscope slide and sponge resulting in a pressure-transfer of the cells from the membrane to the slide. Alternatively the cells on the filter could also be re-suspended by a centrifugation step. It should be noted that the cells that passed through the filter could also be used.
The membrane is carefully peeled back so as not to disturb the transferred cell button on the microscope slide.
The slide is immersed in fixative for later biological analysis, such as immunostaining analysis with an antibody of interest.

4. Alternative Enrichment Method

The following procedure describes the enrichment method for culturing and expansion of recovered cells. In this alternative enrichment method, the 40% ethanol solution is replaced by a isotonic buffered solution.
The blood is transferred from the collection tube to a suitable centrifuge tube.
The blood collection tube may be washed with a small amount of phosphate-buffered saline (PBS), and added to the centrifugation tube.
Slowly inject into the bottom the centrifugation tube 3.0 mL of Ficoll-Paque gradient centrifugation media at room temperature using the 5 mL syringe with the 12 cm tip.
The blood samples are centrifuged at a speed of 400 g for 30 minutes, using a partial or no brake at the end of the run. Batch size should be determined by the total batch processing time of 30 minutes excluding centrifugation time. It is estimated that the maximum size of a batch should be from 4 to 6 samples.
The mononuclear cell fraction (or buffy coat) is aspirated from the centrifuge tube by immersing the tip of a 5-mL syringe fit with the 10 cm silicon-tubing tip attachment below the level of the buffy coat. Aspirate in a steady manner until a small amount of serum is aspirated. The tip should be lowered slightly and aspiration should continue until once again a small amount of serum is aspirated.
The aspirated buffy coat is added directly onto the membrane, pre-primed with a solution that maintains the integrity and viability of the cell. This solution (called Culture Buffer) may be an isotonic buffered solution containing 10% serum by volume. There may be about 10 mL of this Culture Buffer remaining in the top chamber of the apparatus prior addition of the mononuclear cell fraction. Flush the syringe out by aspirating some of the fluid back into the syringe and back out again.
Filter the contents down to approximately 3 mL remaining in the top chamber. Wash the sample by addition of 10 mL of Culture Buffer and back flushing the membrane. Repeat as necessary until filtration is complete (the filtrate is clear and the flow rate is constant). With some samples the flow rate through the membrane may become very slow necessitating a back flush before the contents have reached the 3 mL mark.
At this point, the enriched fraction may be used for cell culturing purposes by at least two alternative methods:

### Method 1:

After the enriched fraction has been filtered down to about 3 mL mark, the contents within the filtration chamber are re-suspended.
The re-suspended fraction is then aspirated and placed in a receptacle suitable for cell culturing.

### Method 2

After the enriched fraction has been filtered down to about 3 mL mark, slowly filter off the remaining liquid in the filtration chamber until the liquid is just removed from above the membrane, do not allow the membrane to dry out.
The membrane itself is then removed from the apparatus and placed directly into the receptacle for cell culturing containing cell culture media.

### Results

Seeding of Blood Cells with Exogenous Tumor Cells

In this experiment, blinded samples were seeded with 4 to 120 cells. Analysis by immunohistochemial detection showed that over 80% of seeded cells in each sample were recovered. See FIG. 3.

5. Appendices

### Appendix A: Detailed Operation Instructions for the Seeded Enrichment Example

1. Attach a 10 cm and 12 cm silicon tubes to two 5 mL syringes.
2. The blood is transferred from the collection tube to a suitable centrifuge tube.
3. The blood collection tube may be washed with a small amount of phosphate-buffered saline (PBS)<1 mL, and added to the centrifugation tube.
4. Fill the syringe with 3.0 mL of Ficoll-Paque gradient centrifugation media at room temperature. Place tip of 12 cm silicon tube at the bottom of centrifugation tube. Slowly inject the 3.0 mL of Ficoll-Pague into the bottom of the tube.
5. The blood samples are centrifuged at a speed of 400 g for 30 minutes, using a partial or no brake at the end of the run (the centrifuge has horizontal swing-out buckets). Batch size should be determined by the total batch processing time of 30 minutes excluding centrifugation time. It is estimated the maximum size of a batch depending on the speed of the operator should be from 4 to 6 samples every 30 minutes.
6. Place a new membrane into the apparatus. (see FIG. 4).

Prime membrane to remove air from under membrane:

i. Filling top with 10 mL of 40% ethanol
ii. Aspirate (F) approximately 5 mL
iii. Backflush (BK)
iv. Aspirate (F) approximately 2 mL

### V. Backflush (BK)

vi. Aspirate (F) approximately 1 mL
vii. Top up to the 10 mL mark with 40% ethanol

There should be no indication of air leaking into the system.

Note: (F) and (BK) refer to instrument controls.

7. The mononuclear cell fraction (or buffy coat) is aspirated from the centrifuge tube by immersing the tip of a 5-mL syringe fitted with the 10 cm silicon-tubing tip attachment below the level of the buffy coat, and aspirating in a steady manner until a very small amount of serum is aspirated. The tip should be lowered slightly and aspiration should continue until once again a small amount of serum is aspirated. An alternative to holding the tube by hand would be to place it in a stand.

The aspirated buffy coat is added directly onto the membrane with 10 mL 40% ethanol. Flush the syringe out by aspirating some of the fluid back into and back out of the syringe.

8. Filter (F) the contents down to approximately 3 mL remaining in the top chamber. Wash the sample by addition of 10 mL of 40% ethanol and back flushing (BK) the membrane. Repeat as necessary until filtration is complete (the filtrate is clear and the flow rate is constant). With some samples the flow rate through the membrane may become very slow necessitating a back flush before the contents have reached the 3 mL mark.
9. Filter down to about the 1 mL mark and then slowly filter (S) the contents until the liquid is just removed from above the membrane; do not allow the membrane to dry out.
10. After enrichment of the disseminated cancer cells, the cells are deposited on a slide by removing the membrane from the apparatus. A microscope slide is pressed on the sponge such that the membrane is ‘sandwiched’ between glass microscope slide and sponge resulting in a pressure-transfer of the cells from the membrane to the slide. Remove the clamp, and remove the top of the filtration apparatus by lifting straight up. Remove the membrane using a fine pair of tweezers, being careful not to touch the area at the center that contains the cells.

In some cases the membrane will stick to the top of the membrane apparatus, in which case use the tweezers to gently pull the membrane down and away from the top. Extra care is required not to pull the membrane across the top as the cell layer could be smeared by contact with the top piece.

Place the membrane cell side up on a sponge dampened with 60% ethanol and pre-loaded into the provided jig.

Align the membrane so that the membrane is between the 4 posts and butting up to the 2 short posts. The long axis of the membrane will be across the slide.

Place a slide over the membrane as shown in the picture, the label side should face down.

Gently press down on the microscope slide over the center of the sponge for about 5-8 seconds. Release the pressure (see FIG. 5).

Lift the slide off the sponge (the membrane will adhere to the slide). Turn the slide label side up. Carefully peel back the membrane so as not to disturb the transferred cell button on the microscope slide.

11. The slide is immersed in fixative for later immunostaining analysis with an antibody of interest (Using 95% ethanol as the fixative is suggested).
12. Press the (F) control for a few seconds to remove any residue filtrate from the bottom of the membrane support.

### Appendix B—Instrument Controls and Connections

The waste bottle vacuum pump should be turned on a few minutes before the instrument is needed to give time to purge the air from the waste bottle.

Then the pump is turned off before cleaning to allow the waste bottle to reach atmospheric pressure.

The instrument has a button (F) for momentary switch, to be pushed to aspirate filtrate.

The instrument further has a button (BK) for momentary switch, to be pushed to back flush. To prevent air getting into the system this switch should only be pulsed briefly for less than a second. Only back flush when there is liquid above the membrane and after the (F)-button has been used for several seconds.

The instrument further has a button (S) for momentary switch, used to slowly remove filtrate from the system.

### B—Example #2

### Fetal Cells

The human extravillous trophoblast-derived cell line SGUPL-4 is derived by transfection of primary human first trimester extravillous trophoblasts with the early region of SV40. SGHPL-4 cells retain many features of normal extravillous trophoblast, such as expression of cytokeratin-7, BC-1, HLA-G, CD9, hPL and HCG (Choy and Manyonda, 1998; Cartwright et al., 1999, Prefumo et al., 2004b) and behave in the same manner as primary cells (Ganaphthy et al. Hum. Reprod. 21 (5): 1295).

SGHPL4 cell line is therefore the best cellular model for the demonstration of the unique capacity of our technology to isolate circulating fetal cells from a blood sample. Here we show that starting for a blood sample containing five SGHPL-4 cells per ml of blood, the recovery of fetal cells is more than 80%. The purity of the isolated fetal cells is 5% as compared to 0.00005% before the process.

### 1-Protocol for Fetal Cell Isolation:

The following procedure describes the isolation method for fetal cells from a blood sample using the apparatus described in Appendix A and B.
Tune the differential pressure of the apparatus, i.e. between the two compartments, to a value comprises between 40 kPa to 60 kPa for all the following steps with the temperature between 20° C. and 40° C.
Pre-prime the system with the wash solution, solution that maintains the integrity and viability of the cell, i.e. PBS1X.
Add the blood sample (5 mL) directly in the top chamber of the apparatus.
Filter the contents down and wash the sample by addition of 5 mL of Wash Solution. Repeat 5 times this step. At each washing steps do not allow the membrane to dry out.
Remove the membrane from the apparatus and place “cells up” it in an appropriate surface for further treatments:
Fixation of the Isolated cells (for example for Immunofluorescence or FISH): The filter are treated by 1 mL Paraformaldehyde 4% for 10 minutes and then washed 4 times with 1 mL of PBS1X
Culture of the Isolated Cells
   the filter is place in a cell culture dish with appropriate culture medium.
alternatively, the cells present on the filter are resuspended with 1 ml of culture medium and place in a cell culture dish.

At this point, the identification cells of interest, i.e. fetal cells, can be performed by immunofluorescence, FISH or any other methodology used for genetic diagnosis.

### 2. Circulating Blood Sample Preparation

Whole blood sample is collected from the peripheral circulation. The blood is collected in a 15 ml polypropylene tube containing an anticoagulant (heparin, EDTA).

3. Spiking Experiment with SGHPL-4 Cells

SGHPL4 cells, which are considered as fetal cells (vide supra), are harvested according to usual cell biology procedures, e.g. cell containers are washed, detached by trypsinization for a suitable length of time, then collected by centrifugation. The collected cells are suspended in a volume of medium without serum and counting cells is performed using a counting chamber with a cover on the top.

A 5-mL whole blood sample is collected from the peripheral circulation of a healthy volunteer. The blood is collected in a 15 ml polypropylene tube containing an anticoagulant.

SGHPL-4 cells are then added to the blood sample at a known nominal value by serial dilution of the dispersed stock. At every step in the series, and in the final seeded blood sample, the tube is gently mixed for cell dispersal.

### 4. Immunofluorescence Detection of SGHPL4 Cells Isolated on Membrane

The blood sample prepared as described in point 3 is processed following instructions for fetal cells isolation described in the Protocol for fetal cells isolation. At the end of the process, the membrane are removed of the apparatus and treated by 1 mL Paraformaldehyde 4% for 10 minutes and then washed 4 times with 1 mL of PBS1X.
All the following steps are executed at Room Temperature
Add 1 ml of PBS1X, Triton 0.1% onto membrane (side up) for 10 min.
Wash the membrane with 1 ml of PBS1X for 0 min.
Treat the membrane with 1 ml of a solution composed of PBS1X, Gelatin 0.25% for 30 min.
Incubate one hour the membrane with 100 microliter of a solution composed of: PBS1X, Gelatin 0.12% with the anti-SV40 largeT, small t antigen monoclonal antibody (BD Pharmingen, cat n^{o} 554150) at a 1:200 dilution.
Wash 3 times the membrane with 1 ml of PBS1X, 5 minutes each.
Incubate 1 hour the membrane with 100 microliter of a solution composed of PBS1X, Gelatine 0.12% with the secondary fluorescent antibody (Goat anti-mouse CY3, Jackson) at a dilution comprised between 1:50 to 1:200.
Wash 3 times the membrane with 1 ml of PBS1X, 5 min. each.
Add 50 mL of a anti fade solution (VectaShield, Vector Laboratories Inc.) with the fluorescent stain DAPI (4′,6-diamidino-2-phenylindole, SIGMA) and cover the membrane with a appropriate slip (22 mm×32 mm).

### 5. Results

In this experiment, blinded samples were seeded with 5 to 50 SGHPL4 cells per ml of blood. The membranes were treated for immune fluorescence cell detection using a specific antibody directed against an antigen expressed by SGHPL-4 cells and not expressed in leukocyte. In the described example, a mouse anti-SV40 Large T, small t Antigen monoclonal antibody was used. At the end of the protocol, analysis by immunofluorescent detection showed that over 80% of seeded cells in each sample were recovered.

The following table shows the number of the different cells type isolated on the membrane. The cells were counted by observation on fluorescent microscope using appropriate filters. The number of all nucleated cells was counted with filter for DAPI staining, and SGHPL-4 cells were counted with filter for CY3 staining.

| | Total Number of | | |
|---|---|---|---|
| Number of | nucleated Cells On | Number of | |
| SGHPL4 per | the membrane: | SGHPL4 cells on | Recovery |
| mL of blood | Leukocyte and | the membrane | Yield of |
| in a 5 | SGHPL-4 (observed | (observed by | SGHPL-4 |
| mL sample | by DAPI staining) | CY3 staining) | cells |
| 50 | 550 to 700 | 150 to 200 | >80% |
| 5 +/− 2 | 400 to 500 | 20 to 35 | 50 to 100% |

The enrichment of the fetal cells like isolation was calculated. The total number of leukocytes per mL of circulating blood is comprised between 4 to 10 millions. The enrichment of the fetal cells like by the process of the invention is superior to 105, as this can be seen in the following table

| Number of | Theoretical | Experimental | |
|---|---|---|---|
| SGHPL4 per | Ratio of | Ratio of | Enrich- |
| ml in a | SGHPL4/leukocytes | SGHPL4/leukocytes | ment of |
| 5 ml sample | before the Process | after the Process | SGHPL4 |
| 5 | 1 out of 2 × 10⁶ | 1 out of 20 | >10⁵ |
| | (0.00005%) | (5%) | |

### 6. FISH Analysis on SGHPL-4 Cells Isolated on Membranes

For this analysis, blood samples are collected from peripheral circulation of women healthy volunteer. Blinded samples were seeded with 100 SGHPL4 cells per ml. The SGIPL4 cells are XY, the leukocytes from women healthy volunteer are XX.

The blood sample prepared as described in point 3 is processed following instructions for fetal cells isolation described in the Protocol for fetal cells isolation. At the end of fetal cells isolation process, membranes are removed from the apparatus and treated by 1 mL Paraformaldehyde 4% for 10 minutes and then washed four times with 1 mL of PBS1X.

FISH experiments are performed by following the instruction manual for the kit “2 Color X & Y Probe Panel”, OnCellSystem, Catalog #ASXY.

Analysis of the red and green signals with appropriate filter of a fluorescent microscope allows to clearly discriminate XY cells from XX cells. These results demonstrate that multi FISH experiments can be performed on isolated cells on membrane.

### 7. Determination of Optimal Parameters for Fetal Cells Isolation

The isolation of circulating rare cells relies on the viscoelastic properties of leukocytes that allow them to pass through membrane pores size smaller than their diameter. This property is dependant to the differential pressure applied between the two compartments but also to the temperature.

### 8. Detection of Isolated Fetal Cells on Membrane for Genetic Analysis

Fetal cells isolated on membrane could be identified by specific antibody directed against a marker expressed by fetal cells and not expressed in leukocyte. Commercial antibodies can be used to identify trophoblast cells are listed in the following table:

| Antibody | Marker | Sigma |
|---|---|---|
| Anti-cytokeratin 18 | Cytokeratin | Sigma |
| Anti-vimentin | Vimentin filaments | Dako Ltd |
| Anti HLA-DR | HLA-DR | Serotec |
| W6/32 | HLA-Class 1 A, B, C | Dako Ltd |
| Anti-hPL | Human Placental lactogen | Dako Ltd |
| Anti-hCG | Human chorionic gonadotrophin | Dako Ltd |
| Anti-SP1 | Pregnancy specific beta 1 | Dako Ltd |
| | glycoprotein | |
| MAC3 | Macrophage | Dako Ltd |
| Anti-von Willibrand factor | von Willibrand factor | Dako Ltd |
| Alpha1 | Alpha1 homodimer | Biogenesis |
| Alpha3 | Alpha3 homodimer | GibcoBRL |
| Beta1 | Beta 1 homodimer | GibcoBRL |

Alternatively, other antibodies are described in the literature to specifically labeled trophoblast cells as following (PMID: PubMed IDentifier):

| | |
|---|---|
| AC133-2 | applicable as a positive marker for the characterization of all subtypes of |
| | trophoblast and for trophoblast cell lines. PMID: 11504532 |
| Cdkn1c | The IPL and p57(KIP2)/CDKN1C genes are closely linked and coordinately |
| | imprinted, and immunostaining showed that their protein products are co- |
| | expressed in villous cytotrophoblast. PMID: 13129680 |
| Cdx2 | the trophoblast-associated transcription factor, is a trophoblast marker. PMID: |
| | 14990861 |
| | a trophoblast stem cell marker. PMID: 16433625 |
| CHL1 | found to be expressed on the majority of EVT, is an extravillous trophoblast |
| | marker. PMID: 12771237 |
| Cytokeration | greater pancytokeratin immunofluorescence is observed in extravillous |
| | cytotrophoblast cells as compared with villous trophoblast. The most invasive |
| | population of cells of the trophoblast lineage (the extravillous trophoblast) |
| | exhibits a significant reduction in cytokeratin immunofluorescence when |
| | comparisons of healthy and pre-eclamptic pregnancies are made. PMID: |
| | 15287017 |
| | a highly reliable marker for cells of the trophoblast lineage in vitro, trophoblasts |
| | should be identified by the presence of cytokeratin 7 in preference to cytokeratin |
| | 8/18. PMID: 10527816 |
| | Application of immunohistochemical staining for cytokeratin allowed proper |
| | identification of trophoblast. PMID: 8906606 |
| | the different populations of human placental trophoblast express cytokeratins in |
| | developmental, differentiative, and functional specific patterns. These findings |
| | can be useful to distinguish and classify the various trophoblastic populations |
| | and provide a foundation for studying pathological aspects of the trophoblast. |
| | PMID: 7539466 |
| Cytokeratin-7 | an accurate intracellular marker with which to assess the purity of human |
| (CK7) | placental villous trophoblast cells by flow cytometry. PMID: 15087219 |
| Dlx3 | initially expressed in ectoplacental cone cells and chorionic plate, and later in the |
| | labyrinthine trophoblast of the chorioallantoic placenta. PMID: 9874789 |
| FD0161G | the extra-villous trophoblast marker and could be used as a specific probe for |
| | extra-villous trophoblast in decidual tissue. PMID: 3301747 |
| Gcm1 (glial cells | a subset of trophoblast cells in the basal layer of the chorion that express the |
| missing 1) | Gcm1 transcription factor. PMID: 16916377 |
| | a marker of differentiated labyrinthine trophoblasts. PMID: 16433625 |
| | GCM1 protein expression studies demonstrated that the transcription factor was |
| | present mainly within the nuclei of a subset of cytotrophoblast cells, consistent |
| | with its role as a transcription factor. PMID: 15135239 |
| | encoding the transcription factor glial cells missing-1 (Gcm1), is expressed in |
| | small clusters of chorionic trophoblast cells at the flat chorionic plate stage and |
| | at sites of chorioallantoic folding and extension when morphogenesis begins. |
| | PMID: 10888880 |
| H315 | a trophoblast marker which reacts with placental-type alkaline phosphatase |
| | (PLAP) associated with the cell-membrane of the syncytiotrophoblast. PMID: |
| | 2590397 |
| | a trophoblast-specific marker. PMID: 3500181 |
| | reacting against a specific antigen present on the surface of fetal trophoblastic |
| | cells. PMID: 3510966 |
| | identifies a trophoblast-specific cell-surface antigen and strongly stained both |
| | placental villous trophoblast and the cytotrophoblastic layer of amniochorion. |
| | PMID: 6312818 |
| | H315 and H316 showed comparable staining of placental villous |
| | syncytiotrophoblast and cytotrophoblast and were also able to distinguish |
| | subpopulations of nonvillous trophoblast in the placental bed, including |
| | perivascular and endovascular trophoblastic cells as well as cytotrophoblastic |
| | elements within the decidua and myometrium. PMID: 6197884 |
| | reacted predominantly with normal placental trophoblast and with lymphocytic |
| | cells, as well as with most transformed or neoplastic cultured cell lines. PMID: 7118296 |
| hCG (human | a hormone synthesized by trophoblast cells. PMID: 15570553 |
| chorionic | marker for the differentiation process of cytotrophoblast cells. PMID: 15852231 |
| gonadotropin) | marker for the differentiation process of trophoblast cells to |
| | syncytialtrophoblasts. PMID: 12942243, PMID: 12820356 |
| | a placental hormone and marker for the differentiation process of cytotrophoblast |
| | cells to syncytial trophoblasts. PMID: 12820352 |
| hCG-beta | a trophoblast marker, is expressed in human 8-cell embryos derived from |
| (Human | tripronucleate zygotes. PMID: 2460490 |
| chorionic | |
| gonadotrophin | |
| beta) | |
| HLA-A/HLA- | HLA-G protein expression in different stages of pregnancy and different |
| B/HLA-C/HLA- | trophoblasts may be related to the controlled invasion of the trophoblast. PMID: |
| G | 16354612 |
| | a nonclassical MHC class I antigen that has been shown to be a specific marker |
| | for normal intermediate trophoblast (IT), can serve as a useful marker in the |
| | differential diagnosis of these lesions. PMID: 12131159 |
| | HLA-G expression in extravillous trophoblasts is induced in an autonomous |
| | manner, independently of embryonic development, and may be an integral part |
| | of placental development allowing its tolerance from maternal immune system. |
| | PMID: 11137214 |
| | It has a tissue-specific expression in trophoblast, where the products of HLA-A, -B |
| | and -C classical genes are absent. PMID: 7583772 |
| | HLA-A, B, C was employed to discriminate intermediate trophoblasts (Its) from |
| | cytotrophoblasts (CTs). PMID: 2584815 |
| hPL (human | marker for the differentiation process of trophoblast cells to syncytial |
| placental | trophoblasts. PMID: 12942243 |
| lactogen) | |
| Inhibin A | Maternal serum inhibin A levels are a marker of a viable trophoblast in |
| | incomplete and complete miscarriage. PMID: 12590643 |
| Integrins | alpha5 integrin mediates binding of human trophoblasts to fibronectin and is |
| | implicated in the regulation of trophoblast migration. PMID: 15846213 |
| | interaction with fibronectin through integrin alpha5 plays an important role in |
| | human extravillous trophoblast invasion. PMID: 17027088 |
| | Integrins display dynamic temporal and spatial patterns of expression by the |
| | trophoblast cells during early pregnancy in humans. PMID: 15255377 |
| | Direct contact between trophoblasts and endothelial cells increases the |
| | expression of trophoblast beta1 integrin. PMID: 15189562 |
| | integrin, alphaIIbbeta3, plays a key role in trophoblast adhesion to fibronectin |
| | during mouse peri-implantation development. In vivo, alphaIIb was highly |
| | expressed by invasive trophoblast cells in the ectoplacental cone and trophoblast |
| | giant cells of the parietal yolk sac. PMID: 15031111 |
| | the alpha 7 beta 1 integrin is expressed by trophoblast cells and acts as receptor |
| | for several isoforms of laminin during implantation. PMID: 11784026 |
| | Villous trophoblast from first trimester and term placenta expresses the integrin |
| | subunits alpha 6 and beta 4, as monitored by immunohistochemistry. PMID: |
| | 7685095 |
| | the expression of a alpha 5 integrin subunit on cytotrophoblastic cell surfaces is |
| | correlated with the appearance of an invasive phenotype. PMID: 8288018 |
| M30 | superior to the TUNEL reaction as a marker for the detection of trophoblast |
| | apoptosis since it is easier to handle, more specific for apoptosis and less prone |
| | to artifacts. PMID: 11162351, PMID: 16077948, PMID: 12456208 |
| Mash2 | the spongiotrophoblast marker. PMID: 16966370, PMID: 15901283 |
| | immunoreactive Mash-2 protein was localized predominantly to the cytoplasm of |
| | human cytotrophoblasts. PMID: 12917334 |
| | trophoblast-specific transcription factors. PMID: 12842421 |
| | may serve as a hypoxia-induced transcription factor that prevents differentiation |
| | to syncytiotrophoblast and aromatase induction in human trophoblast cultured |
| | under low O2 conditions. PMID: 11043580 |
| | Mash-2 expression begins during preimplantation development, but is restricted |
| | to trophoblasts after the blastocyst stage. Within the trophoblast lineage, Mash-2 |
| | transcripts are first expressed in the ectoplacental cone and chorion, but not in |
| | terminally differentiated trophoblast giant cells. After day 8.5 of gestation, |
| | Mash-2 expression becomes further restricted to focal sites within the |
| | spongiotrophoblast and labyrinth. PMID: 9291577 |
| | a mammalian member of the achaete-scute family which encodes basic-helix- |
| | loop-helix transcription factors and is strongly expressed in the extraembryonic |
| | trophoblast lineage. PMID: 8090202 |
| MNF116 | for trophoblast cell identification, is a trophoblast marker. PMID: 12848643 |
| | identified, as expected, syncytial giant cells and mononuclear trophoblasts within |
| | the placental bed and glandular epithelial cells throughout the uterus, but also |
| | cross-react with epitopes expressed in cells other than giant trophoblastic cells |
| | and mononuclear trophoblasts in the uterus and, thus, caution has to be used |
| | when such antibodies are used for the diagnostic characterization of tissues |
| | related to the placental bed. PMID: 8575730 |
| NDOG1/NDOG2 | NDOG1 stained chorionic syncytiotrophoblast but not villous cytotrophoblast |
| | and also did not react with any cytotrophoblastic elements in the placental bed. |
| | NDOG1 distinguished these different subpopulations of trophoblast as early as |
| | 13 to 15 days after ovulation. PMID: 6197884 |
| OKT9 | reacted only with trophoblast of placental chorionic villi and did not react with |
| | any nonvillous cytotrophoblast population. PMID: 6312818 |
| PAI-1 | an immunocytochemical marker of invading trophoblasts. PMID: 2473276 |
| (plasminogen | plays a key role in the regulation of fibrinolysis and cellular invasion by virtue of |
| activator | suppression of plasminogen activator function. PMID: 12398812 |
| inhibitor-1) | present in villous syncytiotrophoblasts and co-localized focally with fibrin-type |
| | fibrinoid on the surface of the chorionic villi. Basal plate and placental bed |
| | extravillous interstitial trophoblasts, as well as vascular trophoblasts, were also |
| | PAI-1 positive. PAI-1 defines specific extravillous invasive trophoblasts within |
| | the maternal decidua. PMID: 11095924 |
| Placental | a trophoblast cell differentiation marker. PMID: 15685636 |
| Lactogen (PL-1, | Trophoblast giant cells release two types of PLs in vitro; a high-molecular- |
| PL-2) | weight lactogen, PL-1, and a low-molecular-weight lactogen, PL-2. PMID: |
| | 3972167 |
| | PLP-A/PLP-B/PLP-C/PLP-D/PLP-E/PLP-F/PLP-L/PLP-M/PLP-N |
| | PLP-A was expressed in both trophoblast giant cells and spongiotrophoblast |
| | cells, whereas PLP-B was expressed in decidual and spongiotrophoblast cells. |
| | PMID: 9472921 |
| | PLP-L and PLP-M are most highly expressed in invasive trophoblast cells lining |
| | the central placental vessel as markers of invasive trophoblasts in the rat. PMID: |
| | 10906059 |
| | Expression of PLP-N mRNA was restricted to migratory trophoblast cells. |
| | PMID: 14656203 |
| | PLP-A, PLP-L and PLP-M are synthesized by both interstitial and endovascular |
| | rat trophoblast cells. PMID: 12885563 |
| | PLP-A is a novel pregnancy- and trophoblast cell-specific cytokine. PMID: |
| | 12850282 |
| | In the mouse, PLF-RP was expressed in the trophoblast giant cell layer of the |
| | midgestation chorioallantoic and choriovitelline placentas and, during later |
| | gestation, in the trophoblast giant cell and spongiotrophoblast layers within the |
| | junctional zone of the mouse chorioallantoic placenta. In the mouse, PLP-F is an |
| | exclusive product of the spongiotrophoblast layer, whereas in the rat, trophoblast |
| | giant cells were found to be the major source of PLP-F, with a lesser contribution |
| | from spongiotrophoblast cells late in gestation. PMID: 10657001 |
| | PLP-A was specifically localized to giant and spongiotrophoblast cells of the |
| | junctional zone. PMID: 2667962 |
| | PLP-C is a major secretory protein produced by spongiotrophoblast cells during |
| | the second half of gestation. PMID: 2036977 |
| | PLP-C mRNA was specifically expressed by spongiotrophoblast cells and some |
| | trophoblast giant cells in the junctional zone region of rat chorioallantoic |
| | placenta. PMID: 1744098 |
| | PLP-A, PLP-B and PLP-C are expressed in distinct cell- and temporal-specific |
| | patterns and can be used to monitor the state of differentiation of rat trophoblast |
| | cells. PMID: 8290493 |
| | PLP-D mRNA was specifically expressed in spongiotrophoblast cells and |
| | trophoblast giant cells of the placental junctional zone. PMID: 8756556 |
| | PLP-Cv is a unique gene structure, and displaying a trophoblast-specific pattern |
| | of transcriptional activation. PMID: 8895375 |
| | Expression of PLP-E is restricted to the trophoblast giant cells, whereas PLP-F is |
| | synthesized only in the spongiotrophoblasts. PMID: 9389541 |
| SBU-1 | an excellent marker for trophoblast uninucleate cells from placenta of sheep at |
| | the later stages of pregnancy. PMID: 1692053 |
| SP-1 | a trophoblast-specific beta 1-glycoprotein. PMID: 2450546, PMID: 3675636, |
| | PMID: 2422727 |
| | In syncytiotrophoblast, SP-1 was expressed in normal pregnancy and |
| | unexpressed in spontaneous abortion. PMID: 9589941 |
| | HCG and SP-1 are equally well suited for the serial evaluation of trophoblast |
| | function in early pregnancy. PMID: 6984404 |
| | a good, additional parameter for the assessment of the trophoblast function. |
| | PMID: 94488 |
| | TA1/TA2 (trophoblast antigens) |
| | expressed on trophoblast membrane. PMID: 6378769, PMID: 3073224 |
| | Tfeb |
| | the chorionic trophoblast marker. PMID: 15987772 |
| | expressed at low levels in the embryo but at high levels in the labyrinthine |
| | trophoblast cells of the placenta, plays a critical role in the signal transduction |
| | processes required for normal vascularization of the placenta. PMID: 9806910 |
| | Troma 1 and CAM 5.2 |
| | a histological trophoblast marker in normal pregnancy and trophoblastic disease. |
| | PMID: 3009660, PMID: 2433238 |
| | Troma 1, a rat monoclonal antibody, was used as a trophoblast marker in |
| | immunohistochemical studies. PMID: 3001198, PMID: 3902998 |
| | Troma1 is a rat monoclonal antibody and can be utilized as a trophoblast marker. |
| | PMID: 2584815. PMID: 6352374 |

1. A method of separating multiple natural biological objects in a solution, wherein the natural biological objects are composed of at least a natural lower viscoelastic biological object type and a natural higher viscoelastic biological object type, and the natural lower viscoelastic biological objects have lower viscoelastic properties than the natural higher viscoelastic biological objects, wherein the natural lower viscoelastic biological objects are tumoral or cancer cells, the method comprising:
a filtration step of the solution wherein:
the membrane is porous and the diameter of the pores is less than the diameter of the natural lower viscoelastic biological objects and also less than the diameter of a portion of the natural higher viscoelastic biological objects, and allowing the natural higher viscoelastic biological objects to pass through the membrane while retaining the natural lower viscoelastic biological objects above the membrane, and
a controlled force is applied, which is kept lower than the predetermined force needed to force the natural lower viscoelastic biological objects to pass through the membrane, and which is higher than or equal to the predetermined force needed to force the natural higher viscoelastic biological objects to pass through the holes, and
a recovery step wherein the separated natural lower viscoelastic biological objects are recovered above or onto the membrane and/or the separated natural higher viscoelastic biological objects are recovered in the filtrate.

2. The method according to claim 2, wherein the controlled force corresponds to a force resulting from a differential pressure comprised between 20 and 190 kPascals, advantageously between 40 and 60 kpascals, more advantageously between 45 and 55 kPascals, and the average diameter of the pores is comprised between 3 μm and 15 μm, advantageously between 8 and 10 μm, thus allowing to recover the natural lower viscoelastic biological objects above the membrane.

3. The method according to claim 1 or 2, wherein the multiple natural biological objects are at least two cell types.

4. The method according to claim 3, wherein the cell types which are recovered are viable cells.

5. The method according to anyone of claims 3 to 4, wherein the solution containing the cell types is a mononuclear cell fraction which results from a centrifugation step of a blood sample.

6. The method according to anyone of claims 3 to 5, wherein one of the cell types is a tumoral or cancer cell type.

7. The method according to anyone of the claims 1 to 6, wherein during filtration step, the temperature is comprised between 20° C. and 40° C.

8. The method according to anyone of the claims 1 to 7, wherein the membrane is a polycarbonate membrane.
